SEQUENCE LISTING (96 sequences)

SEQ ID NO: 1 - protein, 349 aa, Mycobacterium tuberculosis
QAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADD

SEQ ID NO: 2 - protein, 8 aa, Mycobacterium tuberculosis
ITQDLLDR

SEQ ID NO: 3 - protein, 8 aa, Mycobacterium tuberculosis
VVDWLVDK

SEQ ID NO: 4 - protein, 9 aa, Mycobacterium tuberculosis
MPAVTDLVK

SEQ ID NO: 5 - protein, 9 aa, Mycobacterium tuberculosis
YTAPEISAR

SEQ ID NO: 6 - protein, 12 aa, Mycobacterium tuberculosis
DAGQIAGLNVLR

SEQ ID NO: 7 - protein, 11 aa, Mycobacterium tuberculosis
NPLFLDEQLTR

SEQ ID NO: 8 - protein, 13 aa, Mycobacterium tuberculosis
HMGSDWSIEIDGK

SEQ ID NO: 9 - protein, 13 aa, Mycobacterium tuberculosis
HMGSDWSIEIDGK

SEQ ID NO: 10 - protein, 16 aa, Mycobacterium tuberculosis
IVNEPTAAALAYGLDK

SEQ ID NO: 11 - protein, 16 aa, Mycobacterium tuberculosis
ATSGDNHLGGDDWDQR

SEQ ID NO: 12 - protein, 17 aa, Mycobacterium tuberculosis
DVLLLDVTPLSLGIETK

SEQ ID NO: 13 - protein, 20 aa, Mycobacterium tuberculosis
GVNPDEVVAVGAALQAGVLK

SEQ ID NO: 14 - protein, 51 aa, Mycobacterium tuberculosis
ARAGHTALRRGGPDVADPQSPAMHRQRGDDRGVRRAAGGRGSAAVAAGRAQ

SEQ ID NO: 15 - protein, 36 aa, Mycobacterium tuberculosis
PGSARDAGAGAVTRLTLHLAYPDTLATRKQGGALEC

SEQ ID NO: 16 - protein, 4 aa, Mycobacterium tuberculosis
HSHV

SEQ ID NO: 17 - protein, 51 aa, Mycobacterium tuberculosis
SARWQSANPCVGTRDDGARARTNAESPGNSDGSGARPGPTANSGPGERPGL

SEQ ID NO: 18 - protein, 2 aa, Mycobacterium tuberculosis
SK

SEQ ID NO: 19 - protein, 105 aa, Mycobacterium tuberculosis
WRAPIVAKVNIKPLEDKILVQANEAETTTASGLVIPDTAKEKPQEGTVVAVGPGRWDEDGEKRIPLDVAEGDTVIYSKYGGTEIKYNGEEYLILSARDVLAVVSK

SEQ ID NO: 20 - protein, 13 aa, Mycobacterium tuberculosis
SVFRPGDPRAHHG

SEQ ID NO: 21 - protein, 9 aa, Mycobacterium tuberculosis
FPGRHALAD

SEQ ID NO: 22 - protein, 63 aa, Mycobacterium tuberculosis
PCVEEPDEQADRIRRNRASRHGGRHGQAGRHRAGDAGAARPACCAGQGVWRTHGYQRRRHGGT

SEQ ID NO: 23 - protein, 5 aa, Mycobacterium tuberculosis
DRAGR

SEQ ID NO: 24 - protein, 7 aa, Mycobacterium tuberculosis
YGGTEIK

SEQ ID NO: 25 - protein, 7 aa, Mycobacterium tuberculosis
WDEDGEK

SEQ ID NO: 26 - protein, 4 aa, Mycobacterium tuberculosis
AHHG

SEQ ID NO: 27 - protein, 4 aa, Mycobacterium tuberculosis
FPGR

SEQ ID NO: 28 - protein, 9 aa, Mycobacterium tuberculosis
VNIKPLEDK

SEQ ID NO: 29 - protein, 12 aa, Mycobacterium tuberculosis
YNGEEYLILSAR

SEQ ID NO: 30 - protein, 15 aa, Mycobacterium tuberculosis
EKPQEGTVVAVGPGR

SEQ ID NO: 31 - protein, 15 aa, Mycobacterium tuberculosis
IPLDVAEGDTVIYSK

SEQ ID NO: 32 - protein, 23 aa, Mycobacterium tuberculosis
ILVQANEAETTTASGLVIPDTAK

SEQ ID NO: 33 - protein, 136 aa, Mycobacterium tuberculosis
DAAADPAAAAGDVVVQPGGRHRRRQPSRRGSRADGPARHQSAVEPSAGWWIRPQRGRGPAARGVATWRRWVVDPHAADVSADRKAGCPLGDAGGCCRIVGDGWRRSGGCGSDGPGCAIRRLHQAGSGRAGTARAGA

SEQ ID NO: 34 - protein, 42 aa, Mycobacterium tuberculosis
RRRRGRLGRRGRLVSSRNDNRLPGHPGRKTCQHFGEEGKERK

SEQ ID NO: 35 - protein, 102 aa, Mycobacterium tuberculosis
SSMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 36 - protein, 11 aa, Mycobacterium tuberculosis
PANTKRNGAKT

SEQ ID NO: 37 - protein, 34 aa, Mycobacterium tuberculosis
QSSSGISRVSRPRQAQSREMSRPFIPSLTRGSSP

SEQ ID NO: 38 - protein, 19 aa, Mycobacterium tuberculosis
PSSQRPGAVAVRRRTRVSS

SEQ ID NO: 39 - protein, 8 aa, Mycobacterium tuberculosis
QAGVQYSR

SEQ ID NO: 40 - protein, 13 aa, Mycobacterium tuberculosis
GAAGTAAQAAVVR

SEQ ID NO: 41 - protein, 11 aa, Mycobacterium tuberculosis
QELDEISTNIR

SEQ ID NO: 42 - protein, 12 aa, Mycobacterium tuberculosis
EMSRPFIPSLTR

SEQ ID NO: 43 - protein, 14 aa, Mycobacterium tuberculosis
WVVDPHAADVSADR

SEQ ID NO: 44 - protein, 15 aa, Mycobacterium tuberculosis
TDAATLAQEAGNFER

SEQ ID NO: 45 - protein, 18 aa, Mycobacterium tuberculosis
TQIDQVESTAGSLQGQWR

SEQ ID NO: 46 - protein, 18 aa, Mycobacterium tuberculosis
RCAASGWRRWVSTMLRGR

SEQ ID NO: 47 - protein, 18 aa, Mycobacterium tuberculosis
SWRPNAGATANGTSSPNA

SEQ ID NO: 48 - protein, 54 aa, Mycobacterium tuberculosis
CRCKKWCGSPRMRPVSRRHDGRSRCRCFTPWVRWAVCEPDSRAKIPNSAWRRCA

SEQ ID NO: 49 - protein, 68 aa, Mycobacterium tuberculosis
CVPRPMSTTARPSASWVGSHVRWRSRSGRPPGSGRRCAPSGRTPRPRDPKRPRDAAGNVDRRHVLHRS

SEQ ID NO: 50 - protein, 5 aa, Mycobacterium tuberculosis
ATGLC

SEQ ID NO: 51 - protein, 55 aa, Mycobacterium tuberculosis
NPAPITRSPSSRPTDGCRYASMARWSRTRPRRCACRKPVTLQCNIFRWPTWYRIG

SEQ ID NO: 52 - protein, 19 aa, Mycobacterium tuberculosis
SAPRPAPIARSRVKPAITA

SEQ ID NO: 53 - protein, 10 aa, Mycobacterium tuberculosis
LPTPATSSTT

SEQ ID NO: 54 - protein, 10 aa, Mycobacterium tuberculosis
CGRTKTLIRR

SEQ ID NO: 55 - protein, 83 aa, Mycobacterium tuberculosis
RRSRGMSRAIRTKPKSASSRGSAGYRVYLGQRLGVAVFAQRDDHPVTGLEDADERAVISGPVGAHTVAMPLDHYRFTLVDAAD

SEQ ID NO: 56 - protein, 9 aa, Mycobacterium tuberculosis
SGRPPGSGR

SEQ ID NO: 57 - protein, 7 aa, Mycobacterium tuberculosis
WVSTMLR

SEQ ID NO: 58 - protein, 6 aa, Mycobacterium tuberculosis
WPTWYR

SEQ ID NO: 59 - protein, 7 aa, Mycobacterium tuberculosis
CFTPWVR

SEQ ID NO: 60 - protein, 11 aa, Mycobacterium tuberculosis
SPSSRPTDGCR

SEQ ID NO: 61 - protein, 18 aa, Mycobacterium tuberculosis
SWRPNAGATANGTSSPNA

SEQ ID NO: 62 - protein, 2 aa, Mycobacterium tuberculosis
CR

SEQ ID NO: 63 - protein, 11 aa, Homo sapiens
TSIHHTKQAPG

SEQ ID NO: 64 - protein, 19 aa, Homo sapiens
FTVSTTRALTLTLCFSSSP

SEQ ID NO: 65 - protein, 7 aa, Homo sapiens
GHRTRHH

SEQ ID NO: 66 - protein, 8 aa, Homo sapiens
RGRHTRHR

SEQ ID NO: 67 - protein, 8 aa, Homo sapiens
RGYCTRHR

SEQ ID NO: 68 - protein, 266 aa, Homo sapiens
RGRRQGHRQRGRRPRHRQRGRRPRHRQRGRRPRNRQRGRRPRHRQRGRRPGHRQRGRRPGHRQGGRRPRHRQRGRRPGHRQRGRRPGHRQGGRRPGHRQRGRRPGHRQRGRRPGHRQGGRRPGHRQRGRRPGHRQRGRRPWHRQRGRRPGHRQRGRRPRNRQRGRRPRHRQRGRRPGHRQGGCRPGHRQRGRRPGHRQRGRRPGHRQRGRRPKHRQRAVYDIANEDTLQAVANKYTVHNIANEGTVQDITNEGALYDIANGTDKAR

SEQ ID NO: 69 - protein, 8 aa, Homo sapiens
RGRCTRHC

SEQ ID NO: 70 - protein, 7 aa, Homo sapiens
GQRIRHR

SEQ ID NO: 71 - protein, 4 aa, Homo sapiens
LSQE

SEQ ID NO: 72 - protein, 5 aa, Homo sapiens
RPGHR

SEQ ID NO: 73 - protein, 34 aa, Homo sapiens
CNFPTVRIFAKVVSTLYLDSQGSASCTHFSAQIA

SEQ ID NO: 74 - protein, 314 aa, Homo sapiens
MSLASLVNLCQSWKINNLMSTVHSDEAGMLSYFLFEELMRCDKDSMPDGNLSEEEKLFLSYFPLHKFELEQNIKELNTLADQVDTTHELLTKTSLVASSSGAVSGVMNILGLALAPVTAGGSLMLSATGTGLGAAAAITNIVTNVLENRSNSAARDKASRLGPLTTSHEAFGGINWSEIEAAGFCVNKCVKAIQGIKDLHAYQMAKSNSGFMAMVKNFVAKRHIPFWTARGVQRAFEGTTLAMTNGAWVMGAAGAGFLLMKDMSSFLQSWKHLEDEARTETAEELRALAKKLEQELDRLTQHHRHLPQKASQTC

SEQ ID NO: 75 - protein, 4 aa, Homo sapiens
ALAK

SEQ ID NO: 76 - protein, 4 aa, Homo sapiens
GVQR

SEQ ID NO: 77 - protein, 5 aa, Homo sapiens
NFVAK

SEQ ID NO: 78 - protein, 6 aa, Homo sapiens
SNSAAR

SEQ ID NO: 79 - protein, 5 aa, Homo sapiens
HLPQK

SEQ ID NO: 80 - protein, 6 aa, Homo sapiens
AIQGIK

SEQ ID NO: 81 - protein, 6 aa, Homo sapiens
LTQHHR

SEQ ID NO: 82 - protein, 7 aa, Homo sapiens
HLEDGAR

SEQ ID NO: 83 - protein, 7 aa, Homo sapiens
LEQELDR

SEQ ID NO: 84 - protein, 8 aa, Homo sapiens
TETAEELR

SEQ ID NO: 85 - protein, 8 aa, Homo sapiens
FELEQNIK

SEQ ID NO: 86 - protein, 8 aa, Homo sapiens
HIPFWTAR

SEQ ID NO: 87 - protein, 10 aa, Homo sapiens
SNSGFMAMVK

SEQ ID NO: 88 - protein, 9 aa, Homo sapiens
DLHAYQMAK

SEQ ID NO: 89 - protein, 10 aa, Homo sapiens
DMSSFLQSWK

SEQ ID NO: 90 - protein, 10 aa, Homo sapiens
LFLSYFPLHK

SEQ ID NO: 91 - protein, 13 aa, Homo sapiens
DSMPDGNLSEEEK

SEQ ID NO: 92 - protein, 18 aa, Homo sapiens
ELNTLADQVDTTHELLTK

SEQ ID NO: 93 - protein, 27 aa, Homo sapiens
AFEGTTLAMTNGAWVMGAAGAGFLLMK

SEQ ID NO: 94 - protein, 28 aa, Homo sapiens
LGPLTTSHEAFGGINWSEIEAAGFCVNK

SEQ ID NO: 95 - protein, 26 aa, Homo sapiens
INNLMSTVHSDEAGMLSYFLFEELMR

SEQ ID NO: 96 - protein, 57 aa, Homo sapiens
TSLVASSSGAVSGVMNILGLALAPVTAGGSLMLSATGTGLGAAAAITNIVTNVLENR

(19) United States
(12) Patent Application Publication
(10) Pub. No.: US 2006/0210972 A1
(43) Pub. Date: Sep. 21, 2006

(54) Annotation of genome sequences

(75) Inventors: Jonathan Wesley Arthur, Carlingford (AU); Marc Wilkins, Annandale (AU); Mathew Danger Traini, Erskinerville (AU)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: PROTEOME SYSTEMS INTELLECTUAL PROPERTY PTY LTD, North Ryde (AU)

(21) Appl. No.: 10/507,257

(22) PCT Filed: Mar. 13, 2003
(86) PCT No.: PCT/AU03/00300

§ 371 Date: Apr. 27, 2005

(30) Foreign Application Priority Data
Mar. 13, 2002 (AU) PS 1118

(51) Int. Cl. C12Q 1/68; G06F 19/00

(52) U.S. Cl. 435/6; 702/20

(57) ABSTRACT

A method of identifying one or more proteins in an unannotated DNA sequence is disclosed. The method involves dividing the DNA sequence into a plurality of sequence fragments of substantially the same length (about 300 to 5000 base pairs, most typically 1000 to 1050 base pairs. A six frame translation is then performed on each of the DNA sequence fragments to obtain six translated amino acid sequence fragments for each DNA sequence fragment. Each of the translated sequence fragments is subjected to theoretical digestion to obtain a plurality of cleaved peptide sequences. Next experimental empirical data for peptide fragments from a protein digested in the same manner as the theoretical digestion is compared with the theoretical data generated in step for each of the translated sequence fragments to identify one or more translated sequence fragments which include a substantial number of peptides present in the digested protein. The sequence fragment which has the greatest number of theoretical peptide masses correlating to the empirical data indicates the likely location of the protein of interest in the DNA sequence. To avoid problem where the sequence is divided at the site of a protein, the DNA sequence is duplicated and the original and duplicate are split in such a manner that the sequence fragments from the original overlap the cuts in the original genome sequence.

### FIELD OF THE INVENTION

This invention relates to a method of annotation of genome sequences.

### BACKGROUND OF THE INVENTION

Many genomes, including the human genome have now been sequenced. A genome sequence provides a list of bases (A, T, G, C) in the order in which they appear in a length of DNA, however, the sequence per se tells one very little about the genome that is useful and easily or immediately comprehensible. For example in the study of a disease causing bacteria it would be useful in searching for a cure for the disease to determine the location of that part of the bacterium's genome which expressed a particular protein. However, it can be difficult to predict where proteins of interest may be located in a genome sequence. It cannot always be done simply by looking at the sequence per se.

There are a number of known processes for attempting to determine the location of proteins in genome sequence data. The most widely used method for annotation are pattern searching and sequence comparison techniques. One other known method uses computer programs to locate recognisable regions such as start codons and stop codons in a DNA sequence. Other programs attempt to locate proteins by locating regions of high complexity within a DNA sequence which typically indicates the location of a protein.

However, these approaches are far from perfect as in order to implement these programs, various assumptions and hypotheses have to be made about the location of a protein of interest in the DNA sequence, in particular, the potential start and stop positions of the protein. A detection method that requires such assumptions or hypotheses may produce incorrect results if the assumptions/hypotheses are incorrect. For example these procedures are unlikely to locate non-typical sequences, which ironically may be of more interest than other proteins having more typical sequences identified using existing techniques.

Thus, it is one object of the present invention to provide a method for annotating genome sequences, which is hypothesis independent and does not make assumptions for the detection of a protein from nucleic acid sequences.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### SUMMARY OF THE INVENTION

A first broad aspect of the present invention, provides a method of identifying one or more proteins in an unannotated DNA sequence, the method comprising:

(a) dividing the DNA sequence into a plurality of sequence fragments each fragment being of substantially the same length and from about 300 to 5000 bases long;

(b) performing a six frame translation of each of the DNA sequence fragments to obtain six translated amino acid sequence fragments for each DNA sequence fragment;

(c) subjecting each of the translated sequence fragments to theoretical digestion to obtain a plurality of cleaved peptide sequences;

(d) comparing experimental empirical data for peptide fragments from a protein digested in the same manner as the theoretical digestion at step (c) with the theoretical data generated in step (c) for each of the translated sequence fragments to identify one or more translated sequence fragments which include a significant number of peptides present in the digested protein.

Thus the present invention identifies a region of a genome that encodes a protein and optimally defines the open reading frame and therefore the sequence of the protein from the genome. An advantage of the present invention is that no assumptions need to be made about the location of proteins in the DNA sequence data. DNA sequences with non-typical stop and or start codons may be located. The results are hypothesis independent.

Typically the theoretically generated peptide masses are compared to the masses of the peptides experimentally generated by the digested protein and the sequence fragment which has the greatest number of theoretical peptide masses correlating to the empirical data indicates the likely location of the protein of interest in the DNA sequence. The masses of the peptides experimentally generated from the digested protein will typically be determined by mass spectrometry.

It is preferred that the DNA sequence is duplicated and the original and duplicate are split in such a manner that the sequence fragments from the original overlap the cuts in the original genome sequence.

It is important that the sequence fragments are approximately the same length as one another and are sized to equate to the length of a typical protein. Hence, each fragment is, as discussed above, about 300-5000 bases long. Proteins vary in size, most proteins being 10 to 100 kDa i.e. about 300-3000 bases long. Most preferably, the sequence fragments will be around 1000 or 1050 bases long, the latter translating to 350 amino acids which is approximately equivalent to a 33 to 37 kDa protein, which is a common size for a protein.

Using DNA sequences of approximately that length produce about 12 to 20 peptide matches against a background number of matches of commonly around 1 or 2, and up to around 4 for sequences which do not contain a protein.

In a related aspect of the present invention, the step of dividing the DNA sequence and the step of performing the six frame translation can be reversed. Hence, a second broad aspect of the present invention provides a method of identifying one or more proteins in unannotated DNA sequence, the method comprising:

(a) performing a six frame translation of a DNA sequence to provide six translated amino acid sequences;

(b) dividing the six translated amino acid sequences into a plurality of fragments, each fragment comprising 100-1666 amino acids;

(c) subjecting each of the fragments to theoretical digestion to obtain a plurality of cleaved peptide sequences;

(d) comparing experimental empirical data for peptide fragment for peptide fragments from a protein digested in the same manner as the theoretical digestion at step (c) with theoretical data generated in step (c) for each of the fragments to identify one or more fragments which include a significant number of peptides present in the empirically digested protein.

### BRIEF DESCRIPTION OF THE DRAWINGS

A specific embodiment of the present invention will now be described by way of example with reference to the accompanying drawings.

FIG. 1 is a flow chart depicting an overview of the process described in this patent application.

FIGS. 1A to 1E are schematic diagrams illustrating various steps in the method of the present invention.

FIG. 2 is a more detailed flow chart depicting the part of the process involving the segmentation of the genome.

FIG. 3 is a more detailed flow chart depicting the pant of the process involving the translation and theoretical digestion of the genomic segments.

FIG. 4 is a detailed flow chart depicting the part of the process involving the identification of the region of the genome after the peptide mass fingerprinting is complete.

FIG. 5 shows an example of the method in operation using experimental data derived from a spot on a 2D gel of a sample from Mycobacterium tuberculosis The figure identifies the region of the genome coding for this protein as the portion extending over segments 800 to 803. The number of matches or “hits” associated with these segments is distinctly higher than the background number of hits (less than 6).

FIG. 6 shows a detailed view of segment 801 from the search described in FIG. 5 showing the match between specific experimental masses and individual peptides from the theoretical digestion of this segment of the genome. Comparison with the SWISS-PROT database using BLAST shows this region is the coding region for the protein.

FIG. 7 shows a second example of the method in operation on experimental data derived from a different spot from the same sample described in FIG. 5. The figure identifies two potential coding regions (one involving segments 7308 and 7309 and the other involving segments 8290 and 8291). As a number of matches is not substantially above the background, further information is required to confirm this is a coding region.

FIG. 8 shows a detailed view of segment 7309 from the search described in FIG. 7 showing all but one of the peptide matches are located in a contiguous region of amino acids between two stop codons. This confirms this segment is a coding region. Comparison with the SWISS-PROT database using BLAST shows this region is the coding region for the protein.

FIG. 9 shows a detailed view of segment 8290 from the search described in FIG. 7 showing all but one of the peptide hits are located in a contiguous region of amino acids between two stop codons. This confirms this segment is a coding region. Comparison with the SWISS-PROT database using BLAST shows this region is the coding region for the protein.

FIG. 10 shows a detailed view of segment 318 from the search described in FIG. 7 showing all but two of the peptide hits are separated from each other by stop codons. This confirms this segment is not a coding region.

FIG. 11 shows a graph depicting the results of a simulation to demonstrate the effectiveness of the method. On average, for all proteins in Pseudomonas aeruginosa, the best hit, corresponding to the coding region has more peptide hits than the nearest incorrect hit. This distinction is particularly evident in large proteins but decreases as the proteins become smaller.

FIG. 12 is a graph depicting the effect of changing the segment size on the average best and nearest incorrect hits. As the size of the segments increases, the distinction between the two curves increases. The effect on the best hit is limited by the size of the protein. Once the protein is smaller than the size of the segment, there is no longer any effect on the “best hit” curve.

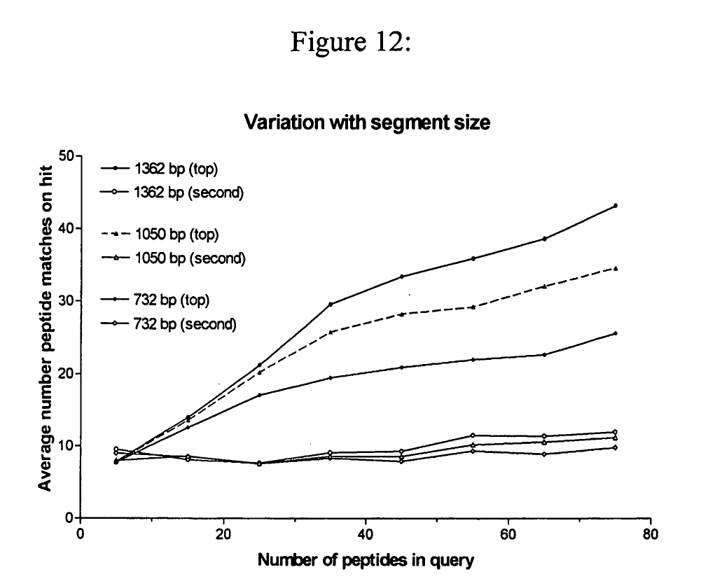

FIG. 13 shows a figure depicting the definition of the best and second best hit. The nearest incorrect hit is the segment having the most hits, when the segments overlapping the best hit are ignored. This is a necessary distinction because adjacent segments to the top hit will often have a large number of hits because the protein sequence is extending across multiple segments.

FIG. 14 shows an example of the application of the method to Homo sapiens. The theoretical digestion of Apolipoprotein L5 (Q9BWW9) was searched against the genomic data from chromosome 22 of H. sapiens. The figure identifies a potential coding region involving segments 36302 and 36303. As there are a number of other matches with similar numbers of hits, further information is required to confirm this as a coding region.

FIG. 15 shows a detailed view of segment 8866 from the search described in FIG. 14 showing the large number of hits is artificial because one experimental mass has matched several, separate points on the segment because this segment contains a repeat region. All matching segments except 36302 and 36303 were similar in that they involved repeat regions.

FIG. 16 is a detailed view of segment 36302 from the search described in FIG. 14 showing the match between specific experimental masses and individual peptides from the theoretical digestion of this segment of the genome. This confirms these segments are a coding region, and comparison with the SWISS-PROT database using BLAST shows this region is the coding region for Q9BWW9.

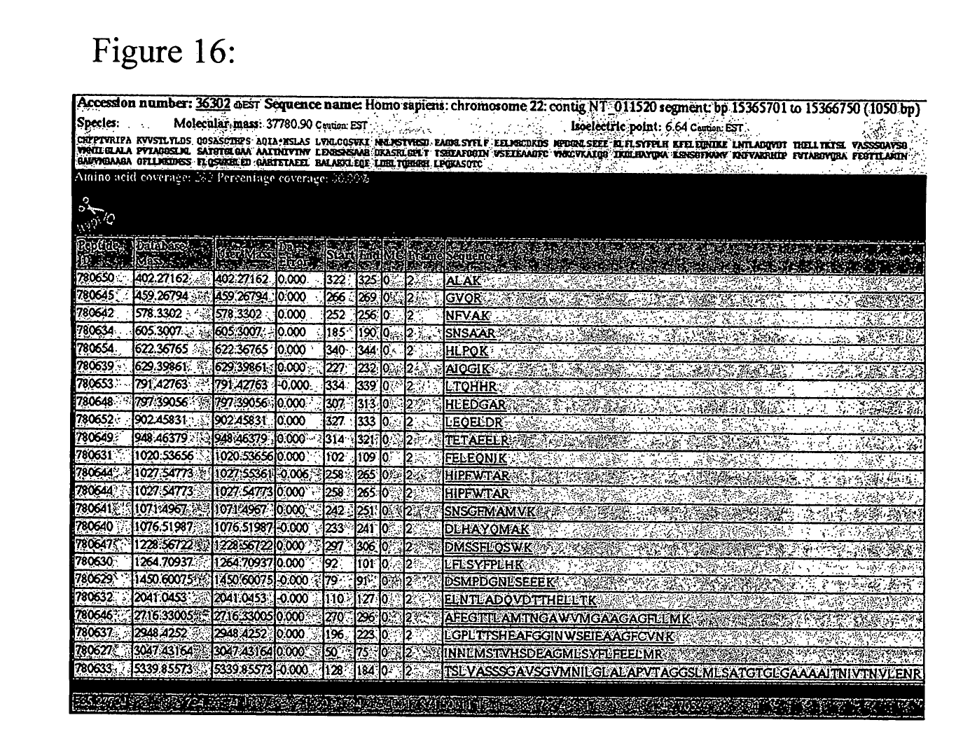

### DETAILED DESCRIPTION OF A PREFERRED EMBODIMENT

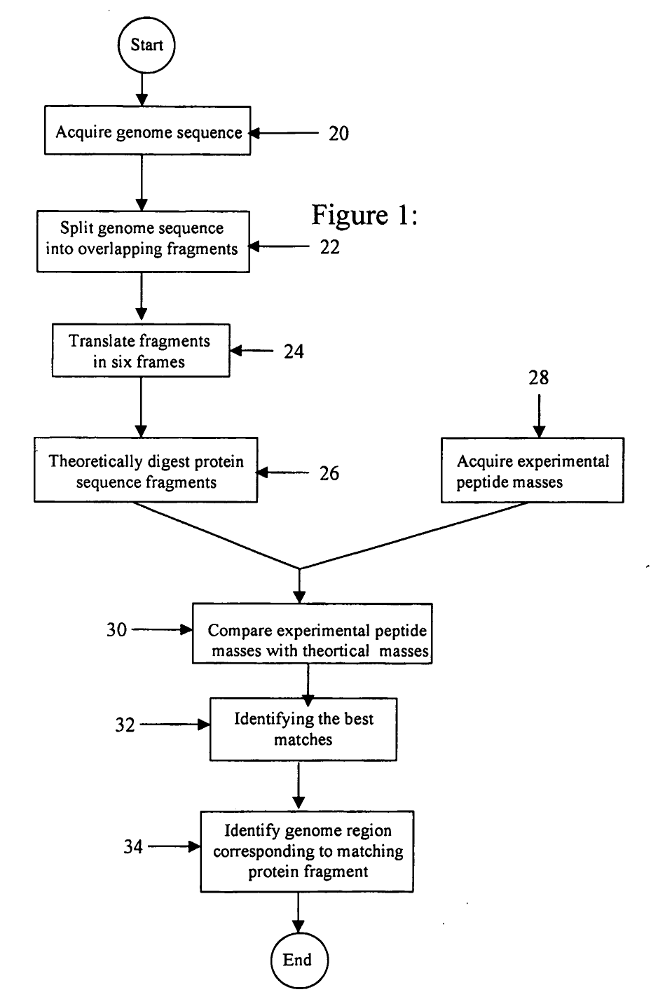

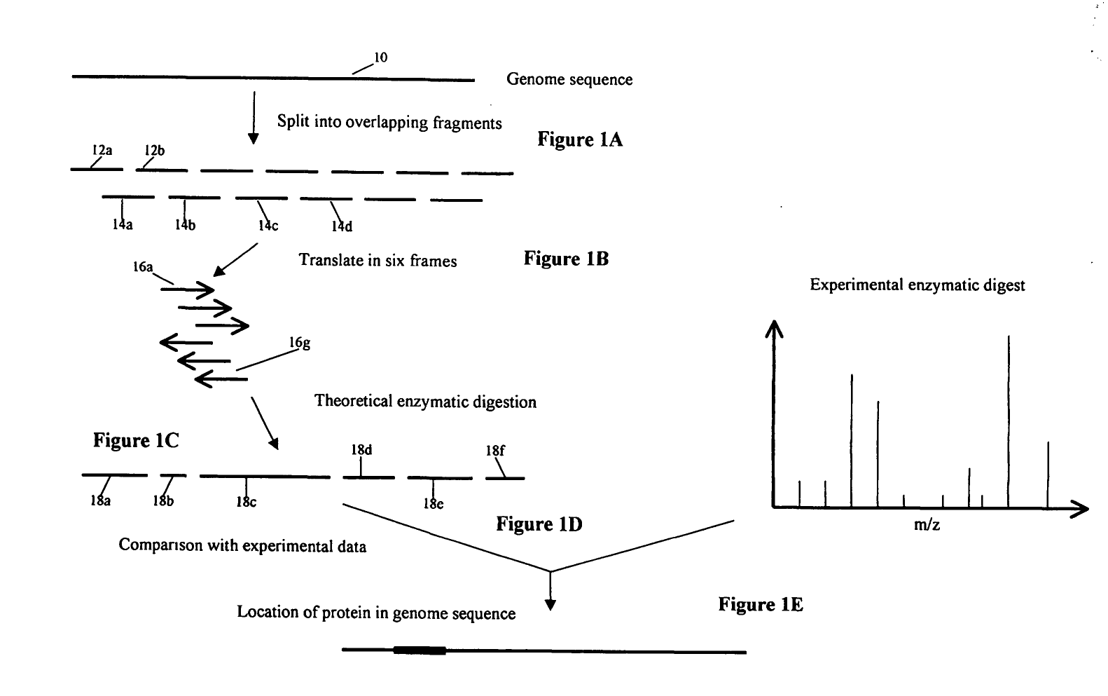

Referring to the drawings, FIG. 1 is a flow chart showing an overview of the method of the present invention. The first step 20 involves the acquiring of a genome sequence. In the next step 22, the genome sequence is split into overlapping fragments. Next at step 24, the fragments are translated in six frames and at step 26 a theoretical digest of the protein sequence fragments generated by the six frame translation is carried out. Step 28 which is independent of the theoretical treatment of the genome sequence shown in boxes 20 to 26 is the acquiring of experimental peptide masses, typically by mass spectrometry. The next step 30, involves the comparison of the experimentally determined peptide masses with the theoretical masses. Step 32 is the process of identifying the best hits, and step 34 is the step of identifying the genome region corresponding to the protein. The process is shown diagrammatically in FIGS. 1A to 1E.

FIG. 1A, shows a genome sequence 10 which is taken and split into a series of shorter genome sequences or sequence fragments 12. Overlapping sequences are preferably provided by duplicating the genome sequence and cleaving the duplicated sequence at locations midway between the breaks in the original sequence so that the sequences (12a,12b . . . , 14a, 14b . . . ) are overlapping as shown in FIG. 1A.

The segments are overlapped to facilitate the process of identifying the region of the genome coding for the protein of interest. In some cases, the peptide masses from the protein of interest could be distributed across two adjacent segments, with a portion of the peptide masses at the end of one segment and a second portion at the start of the next segment. This means the number of peptide masses on each of the two segments will be closer to the background number of random, “noise” matches found on the remaining segments making it harder to identify the hit. However, by using overlapping segments, the peptide t the end of one segment and the start of the next will all be located on the common, overlapping segment. This means the number of peptides on the common, overlapping segment will be further from the background number of random, “noise” matches making it easier to identify this segment as the correct location of the protein-coding region in the genome.

In principle, the overlap is not absolutely necessary for the method to work but it is significant in distinguishing a hit from background “noise”, particularly in the case of relatively small proteins. For example if overlapping were not used and a relatively small protein fell equally between two adjacent segments, only three or four hits might be obtained for each segment. This would not be distinguishable over the background “noise” of typically about 4 hits, so it would not identify the protein. Using overlapping segments, there is a good chance the smaller protein would fall in a single fragment, and the number of hits would be maximised and so facilitate the identification.

Typically, the genome will be cut into sequence fragments which are 1050 bases long. This approximates to 350 amino acids which will be found in a protein of around 33 to 37 kDa which is a common protein size. A bacterium such as Mycobacterium tuberculosis (Tb) will have around 4.4 million bases in its genome. Duplicating and cutting that genome will result in approximately 8400 sequence fragments.

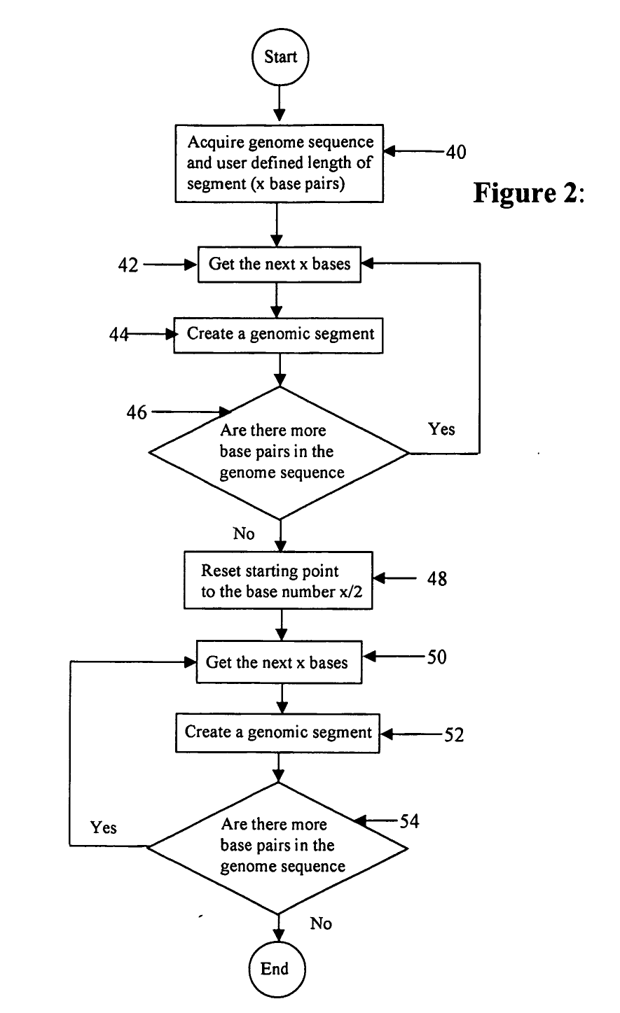

FIG. 2 shows a flow diagram depicting an algorithm for carrying out the part of the process involving segmentation of the genome. The first step 40 involves the acquisition of a genome sequence and the user defined length “x” of segment into which the genome is to be cut, x typically being 1050 base pairs. The first x bases from a starting point at one end of the genome sequence are then acquired at step 42 to create a genomic segment x bases long at step 44. Next at step 46 a check is carried out to see if there are any more base pairs in the genome sequence and if the answer is yes, the next x bases are removed at step 42 again to create a second genomic segment and so on until there are no more base pairs in the genome sequence and the entire sequence has been segmented. When there are no more base pairs in the genome sequence, the algorithm moves to step 48 where a new starting point at base number A is identified, the next x bases from that starting point are then removed at step 50 and used to create a genomics segment, step 52, and the process is repeated, step 55, until there are no more base pairs in the genome sequence. For ease of analysis the first set of segments are numbered 1, 3, 5, . . . n+2, . . . and the second set of fragments overlapping the first are numbered 2, 4, . . . n+1, . . . which ensures that the fragment overlapping two fragments x and x+2 is x+1. This indicates where segments are relative to each other in a readily understandable way and makes it easier to interpret the results.

The genome is segmented to enable easier identification of the protein-coding region of the genome. The genome is segmented into fixed sections, regardless of the length or possible location of the protein coding regions. Hence, the number of background or random matches to the peptide masses is reasonably constant and this then helps to identify the protein coding regions. When the number of matches against a region exceeds the number of random matches on other segments, a protein-coding region is indicated.

If the genome were not segmented, it would be difficult to determine when a concentration of hits was indicative of a protein-coding region. It would be necessary to look for a certain number of hits in a certain length region, but the exact value of these parameters would need to be pre-determined and may affect the results.

Each segment of the genome simulates a protein (the translation of a certain region of a genome). By segmenting, the peptide mass analysis is analogous to peptide mass fingerprinting. This allows the use of a number of existing PMF search engines to do the analysis. Most advantageously, the present invention addresses a very complex problem of mining of genomes with proteomic data but presents the results of this in a way which is completely familiar and highly understandable to the proteomics researcher which does not require the researcher to relearn a new tool or paradigm.

Further, segmenting the genome has advantages in terms of computational performance. In particular, working with a whole genome at once is likely to be demanding in terms of computer memory. Smaller segments can be analysed sequentially and thus require less memory at any particular point in the calculation.

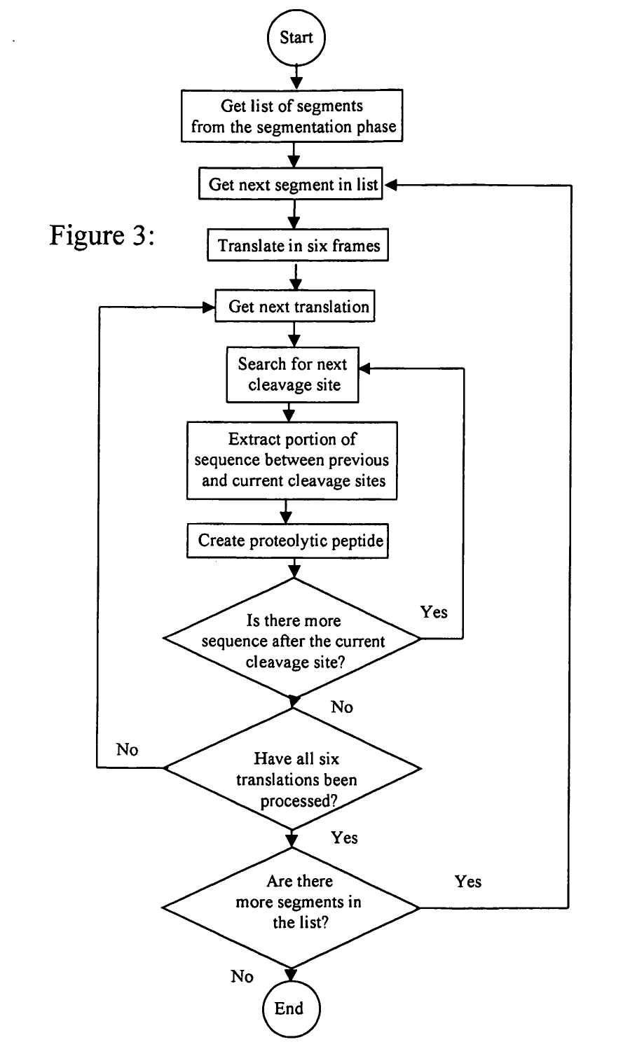

A six frame translation is then carried out on each of the sequence fragments. FIG. 1B schematically illustrates a 6 frame translation carried out on one of the sequence fragments (14d). Six frame translation is a well understood term for the translation of a given nucleotide sequence to the peptide to the peptide sequence in accordance with the universal genetic code, with the translation being done in all three reading frames and in the forward and reverse directions. For each fragment, six virtual proteins are produced. Fragment 14d produces six virtual proteins 16a-16g. Using the M Tuberculosis example referred to above the 8400 sequence fragments become 50,400 virtual proteins, These virtual proteins are then subjected to theoretical digestion according to rules which mimic the action of an endoproteinase enzyme such as trypsin which cut at specific target sites on a target sequence. In a preferred embodiment of the theoretical digest all theoretical peptides which contain a stop codon are removed however the mass of the theoretical protein is calculated from the n terminus of the peptide up to and including the amino acid n terminal to the stop codon. This reduces background noise. This digestion is schematically illustrated in FIG. 1C. Each virtual protein becomes a series of “virtual peptides” and the mass of each virtual peptide is calculated. “Protein” 16g becomes six peptides 18a to 18g. Fewer or more peptides may be produced from each virtual protein. The protein of interest is then subjected to an empirical digestion using the same enzyme and peptide mass data is obtained from mass spectrometry of the peptides expressed by that protein. FIG. 3 is a flow chart depicting part of the process involving the translation of the theoretical digestion of the genomic segments.

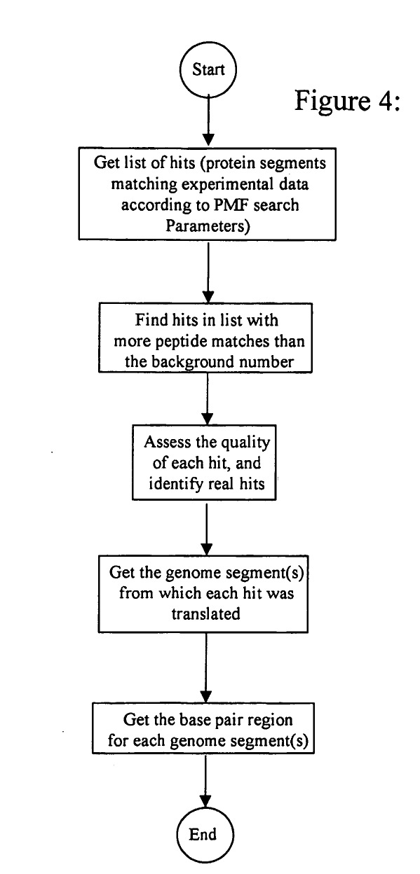

The masses of the various empirically derived peptides are then compared with the theoretical peptide masses produced by theoretical cleavage of the sequence fragments. This is done in a stepwise manner and frame by frame whereby all the empirical peptide masses are matched against all peptides from the first virtual protein and the number of matching peptides (matches or “hits”) is recorded. For each virtual protein, this process is carried out six times, once for each of the amino acid translations. However, the number of matches for each frame is calculated separately and the matches are not summed together. This process is then repeated for the second virtual protein and so on, until it has been carried out for all the virtual proteins. This step is illustrated in FIG. 1D. There is a background number of matches. Typically, each theoretical protein or sequence fragment will produce 1 or 2 matches with a maximum of about 3 or 4 peptides having masses which correlate to masses produced by the actual empirical digest of the protein of interest. The sequence fragment which produced the protein of interest will, in contrast, typically have about 12 to 20 peptide matches with the empirical digest of the protein of interest but is limited by the number of peptides generated empirically. FIG. 4 is a flow chart illustrating this process.

Clearly the relevant part of the genome sequence may have been cut in the original division of the genome sequence, however the overlapping of the original and duplicate genome sequences reduces the risk of this. Even if the protein is split it may still be possible to identify the relevant part of the genome sequence if there are a reasonable number of hits, e.g. 6 to 10, in two adjacent overlapping fragments. The part of the sequence which carries the most peptide masses which match the peptide masses produced by the empirical digestion and has a number of hits which is clearly above the background (noise) level is likely to be that part of the genome which carries the protein of interest. By knowing where the part of the sequence came from this identifies the location of the protein in the genome sequence (FIG. 1E).

### EXAMPLE A (i)

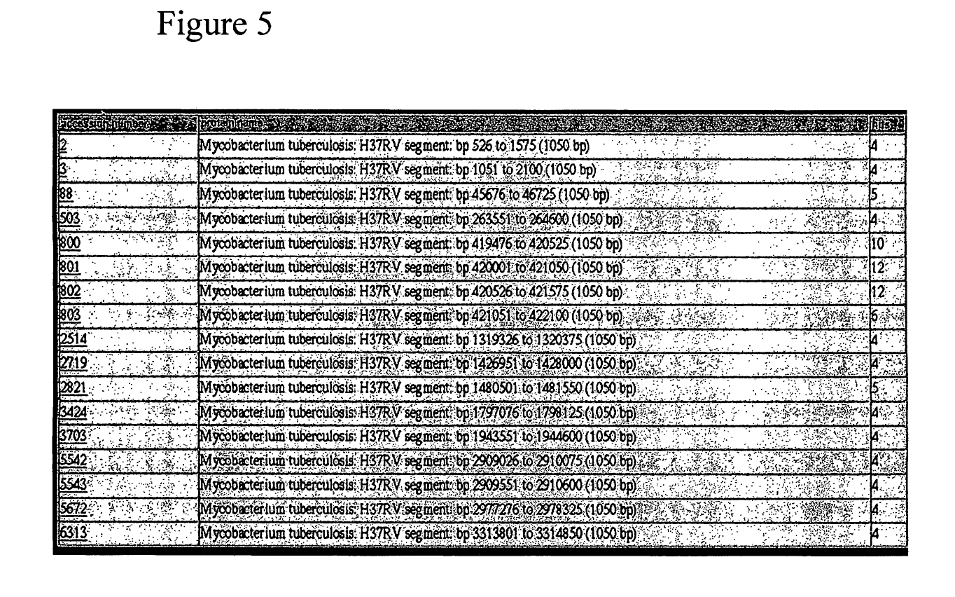

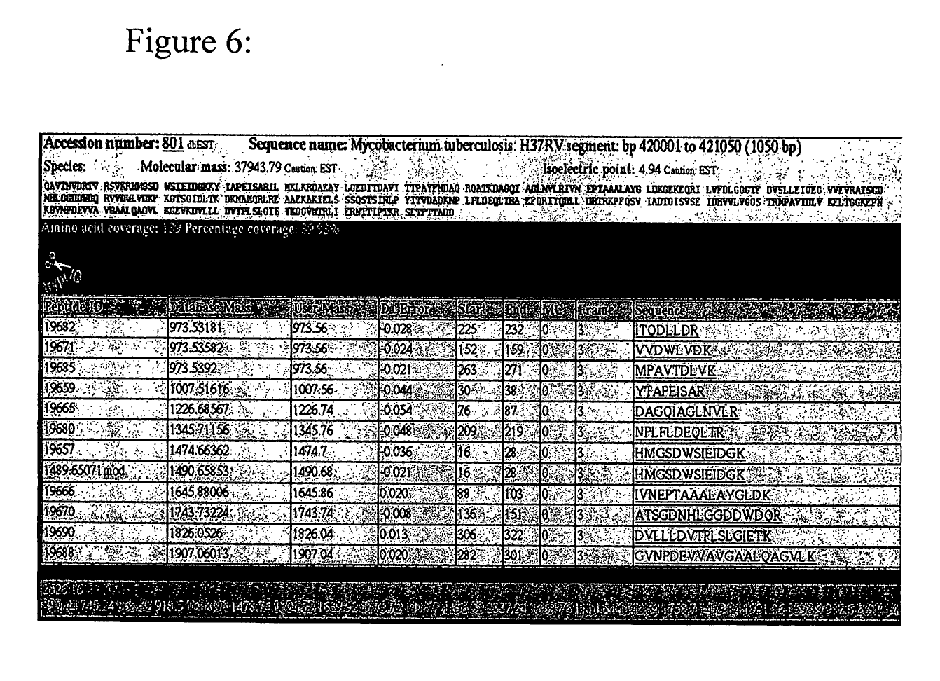

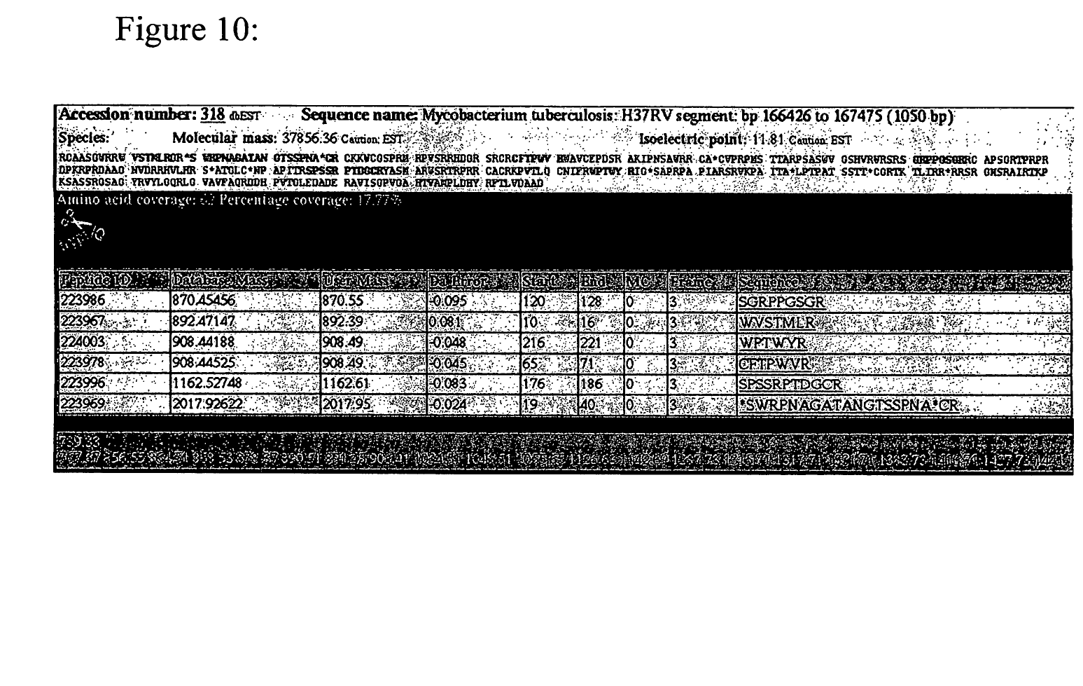

FIGS. 5 to 10 illustrate the results of carrying out the method of the present invention,

A culture of Mycobacterium tuberculosis was used as the source of proteins for experimental analysis. The sample was prepared and the proteins separated using 2D gel electrophoresis. A number of spots were cut from the gel, digested with trypsin, and the peptides resulting from the digestion were analysed with MALDI mass spectrometry. These peaks were analysed using standard peptide mass fingerprinting to identify the proteins contained in each spot,

The genome of M. tuberculosis was segmented into 1050 base pair segments, translated, and theoretically digested using the process described above. The peaks were searched against the genome using the method of the present invention as described above.

The peaks from a first spot were searched with 0.1 Da error tolerance, allowing for cystines to be modified by iodoacetamide and for methionine sulfoxide modifications, and minimum to match of four hits.

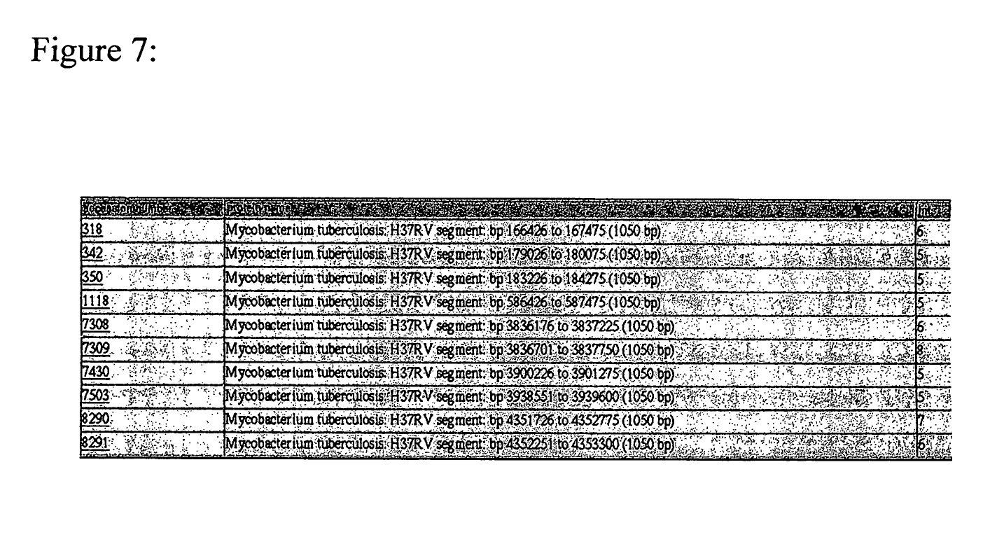

FIG. 5 shows a summary of the results illustrating all the theoretical sequence fragments which produced four hits or more, Four consecutive segments (800-803) received 10, 12, 12, and 6 hits respectively. All other segments had less than 6 hits. This indicates the protein found on the gel matches the region of the genome stretching across these four segments. The protein sequence of segment 801, shown in FIG. 7, was compared to all the proteins in the SWISS-PROT database using BLAST. The protein was thus identified as “Chaperone protein dnaK (P32723)”. This protein of molecular weight 66.7 kDa exactly matches the identification determined by standard peptide mass fingerprinting, indicating that the method described in the patent application correctly identified the region of the genome coding for the protein of interest.

### EXAMPLE A (ii)

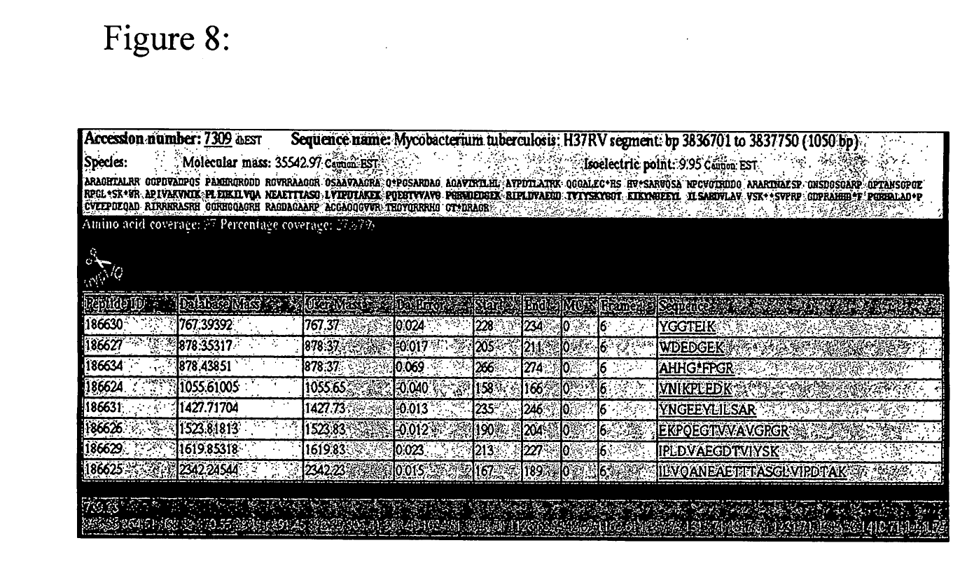

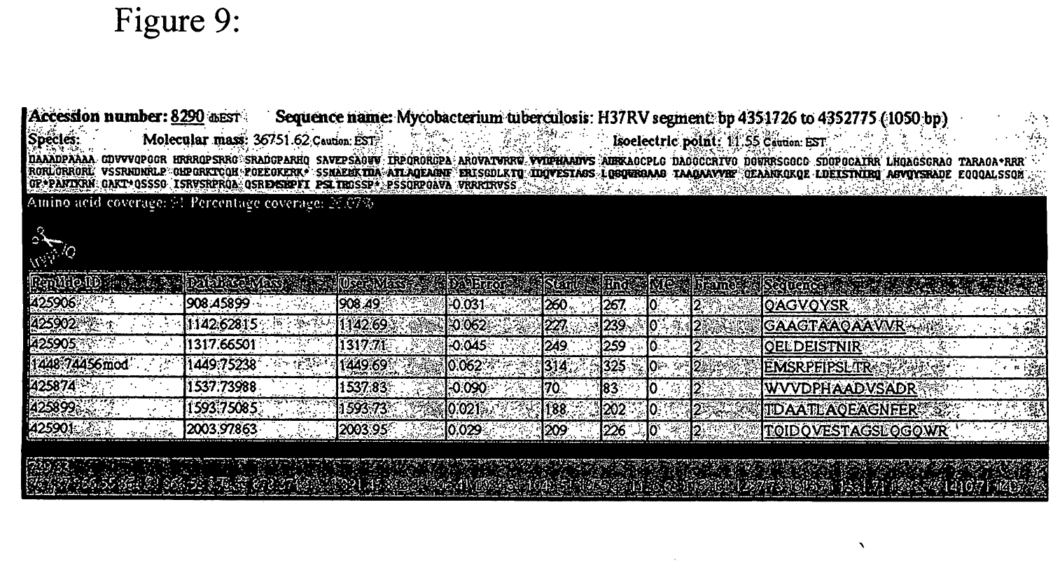

A second spot from the gel was then searched. FIG. 7 is a summary of the results. The peaks from the second spot were searched with the same parameters described above except a value of five hits was used as the minimum to match. Two regions of interest were found. The first involved segments 7308 (6 hits) and 7309 (8 hits), the second involved segments 8290 (7 hits) and 8291 (6 hits). There was one other segment with 6 hits. All the other segments had less than 6 hits. This is illustrated in FIG. 7. The portion of the protein sequence between two stop codons having the most hits was, in each case, submitted to BLAST as described above. The first region, shown in FIG. 8, identified as “10 kDa chaperonin (P09621)” The fact that this is a good result is indicated by the fact that the peptides all occur in a region of consecutive amino acids with no stop codons. Another indicator of a valid result is to check for the presence of initiation methionine. However, it is to be noted that in this case there is no initiation Methionine in this area. This indicates that either there a non-standard start codon being used or that there is an error in the genome sequence. This open reading frame would not have been detected using the standard prior art techniques which demonstrates the usefulness of the approach of the present invention. The second region shown in FIG. 9 identified as a “10 kDa culture filtrate antigen cfp 10 (o69739)”. This clearly does include initiating methionine which neatly defines the open reading frame for the protein.

Both these proteins were found in this spot using standard peptide mass fingerprinting. These proteins did not stand out as clearly as in the previous spot, but were still identifiable. This demonstrates the process described in the patent application can also work when multiple proteins are located in the one spot and when the proteins being searched for are relatively small.

An incorrect hit is shown in FIG. 8 for comparison. Factors which point to it being an incorrect hit are that there is no obvious initiation Methionine present, and there are frequent stop codons present in the reading frame.

### EXAMPLE B

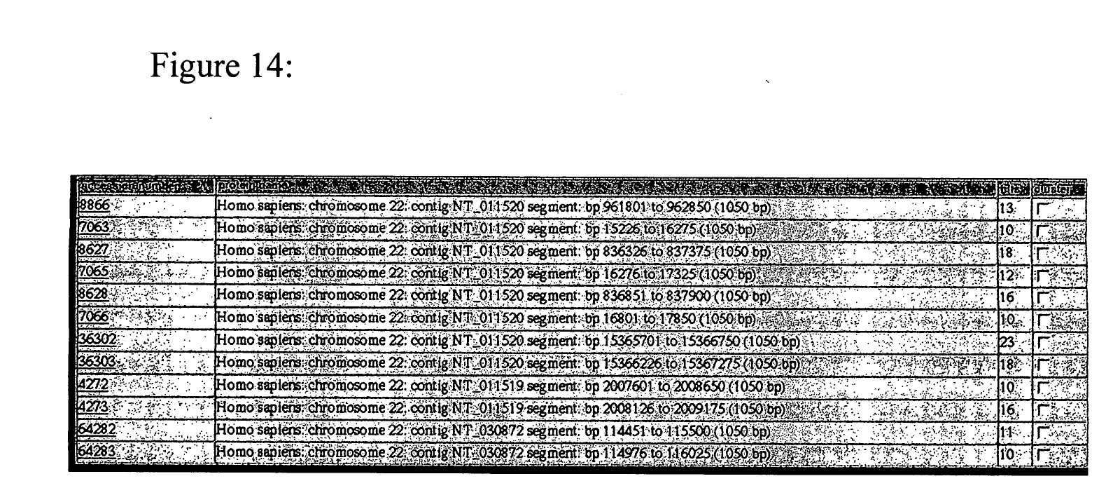

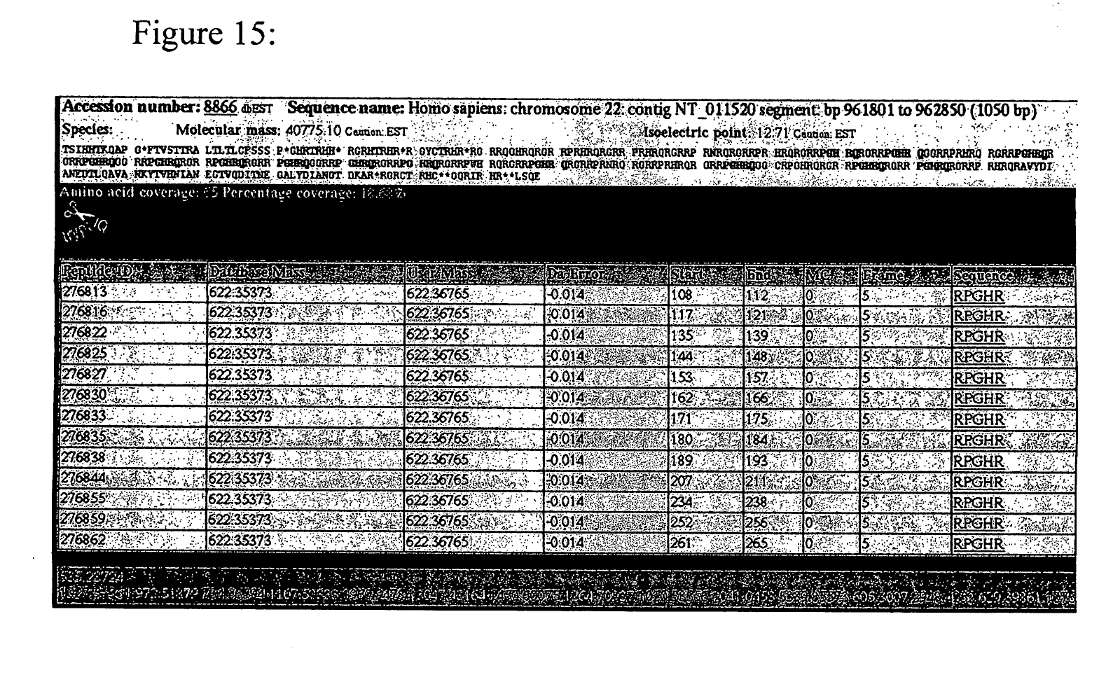

The method can be applied to higher order genomes including the human genome. To demonstrate this the genome sequence of chromosome 22 of Homo sapiens was prepared and searched using the method described above. A theoretical peak list was generated using the sequence of Q9BWW9 (Apolipoprotein L5) known to be located on chromosome 22. This peak list was searched against the genome using the method described in the patent application using an error tolerance of 0.1 Da and a minimum to match of 10. FIG. 14 shows the result of this search. There were 12 hits with between 10 and 23 matches. Examining the details of each of these in turn shows all except two of these hits involve matches to repeat regions in the genome i.e., the same peptide occurs multiple times repeatedly resulting in an artificially high number of matches. This is shown in FIG. 15. The remaining two hits are on overlapping peptides. One of these is shown in FIG. 15. Comparing the sequence of this segment to all the proteins in SWISS-PROT using BLAST identifies the protein as Q9BWW9.

### COMPARATIVE EXAMPLES

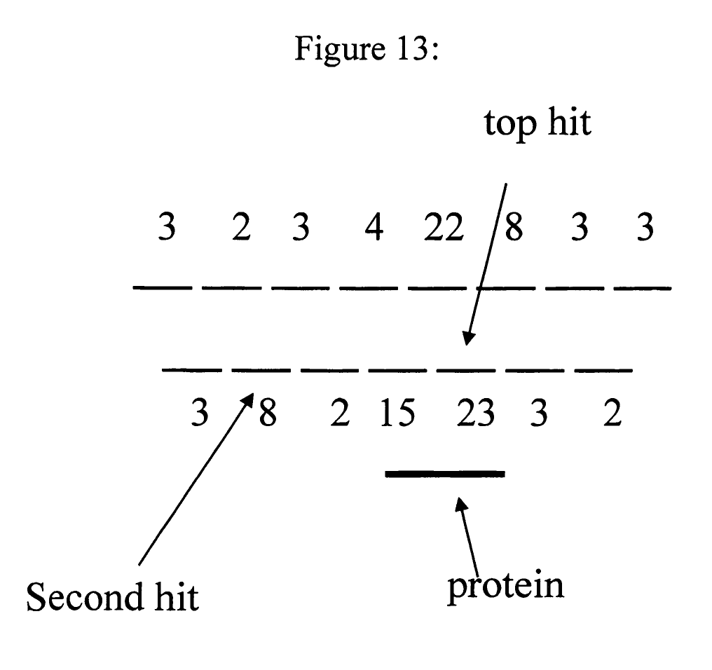

A series of computational simulations were run in order to demonstrate the method and determine the optimum parameters for the method. The simplest simulation involved taking the set of known proteins for Pseudomonas aeruginosa. The set of 773 known proteins was taken from SWISS-PROT. Each protein was theoretically digested according to the cleavage rules of trypsin. Tryptic peptides whose mass was less than 400 Da were discarded, as these masses are not usually seen on a typical MALDI mass spectrum. The remaining tryptic peptides of each protein in turn were searched against the raw genome using the method described in the patent application. The region of the genome coding for the protein was determined by finding the segment with the highest number of matching peptides. The nearest incorrect hit was determined by finding the segment with the next highest number of peptides, excluding those segments connected to the segment with the highest number of peptides through a chain of overlapping segments. This is illustrated in FIG. 13. This allows for the fact that the protein may be longer than one or more segments and thus may have a significant number of hits on adjacent segments.

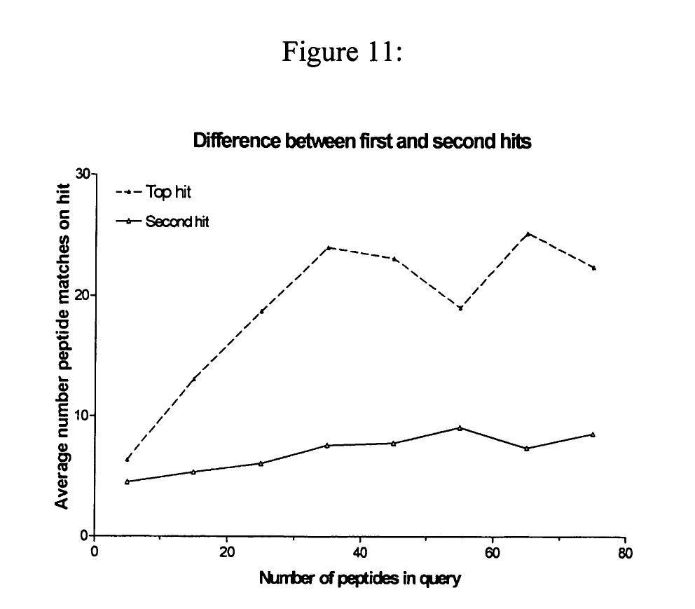

In order to summarise this information, the proteins were binned according to the number of tryptic peptides with mass greater than 400 Da generated from them in a theoretical digestion. The first bin contained all protein with 1 to 10 peptides, the second all proteins with 11 to 20 peptides, etc. The number of matching peptides in the best hit for each of the proteins in the bin was averaged, as was the number of matching peptides in the nearest incorrect hit. These two numbers were plotted as in FIG. 11 to show the difference between the correct hit and the best of the incorrect hits.

The results showed a distinct difference between the best hit and the best of the incorrect hits. The average second best hit has about four to five matching peptides for small query proteins, increasing to around nine to ten matching peptides for larger proteins. For a set of peptides to clearly be identified with a particular region of the genome, they must match more than this number of peptides. This is shown in the figure where the average number of matching peptides in the best hit is significantly higher than the second best hit. For large proteins, the average number of peptide matches approaches 25. This number is limited by the size of the segment as only a certain number of peptides can be expected to fit in the 1050 base pair segment. For smaller proteins, the difference between the first and second hits decreases as there are less peptides in the query sequence, but it can be seen that for all but the smallest proteins, a difference between the two hits is maintained with the average number of matches in the best hit around six to seven.

Several variations on the simulation were done to estimate the effect of different parameters involved in using the method.

1) Increasing the minimum to match, increased the difference between the two curves.

In an application of the method described, the minimum to match should take a value between four and nine, as this is the range for background hits determined in the experiment outlined above. Generally, a high value would be used first to screen out as much background noise as possible. This value would be gradually lowered, if necessary, until a region with a significant matching number of peptides is found.

2) Increasing the size of the segments increases the difference between the two curves. The number of random matches in the second best hit increases slightly, but the number of matches on the best hit increases significantly. A very long segment length is not used because once all query proteins are smaller than the size of the segment no improvement in the obtained and the bigger the segment is the harder it is to locate smaller proteins. In an application of the method described we use 1050 base segments, because this represents a good balance between the two.

3) Changing the composition of the query peak list by adding random peptides has almost no effect on the curves.

In an application of the method described, the peak list is determined by the data extracted from the mass spectrometer. The amount of real peaks and noise peaks is not known in advance.

4) Decreasing the error tolerance for the match between the query masses and the genome masses, increases the difference between the two curves. This is because the query masses are less likely to match another mass in the genome through random chance as the difference in mass tolerated when accepting a match is much smaller.

In an application of the method described, the error tolerance is usually taken in the range of 0.01 to 0.2 Da for experimental masses derived from MALDI mass spectrometry. The value is usually chosen to reflect the accuracy of the technique used to acquire the experimental masses. A typical value is 0.1 Da.

In an application of the method, the peak list used, as input, is the masses of the proteolytic peptides determined by mass spectrometry. The raw spectrum acquired from the mass spectrometer contains many “noise” peaks. Most of these are removed by using a peak-picking algorithm such as the one outlined in Breen et al. (2000, in press) [Breen, E. J., Hopwood, F. G., Williams, K. L., Wilkins, M. R. (2000) Automatic Poisson peak harvesting for high throughput protein identification, Electrophoresis, 21, 2243-2251; Breen E. J., Holstein, W. L., Hopwood, F. G. Smith, P. E., Thomas, M. L., Wilkins, M. R. (2003) Automated peak harvesting of MALDI-MS spectra for high throughput proteomics. Spectroscopy. In press.]

In the simulated testing described above, the peaks used were the masses calculated from the sequence of theoretically cleaved peptides. Masses under 400 Da were excluded because a MALDI mass spectrometer cannot generally measure peptide masses in this range.

The implementation of the methods described in the above examples, assumes the enzyme used to digest the gel spots is trypsin. This is the most common enzyme used experimentally. Thus the theoretical digestion of the segments is also done using the cleavage rules of trypsin.

The method can use any appropriate enzyme to digest the experimental proteins. In this case the theoretical digestion of the genome segments needs to use the cleavage rules for the enzyme to be used in the experimental analysis.

If the experimental analysis is done with multiple enzymes it is possible to use the findings from multiple searches with each of the enzymes to confirm the identification of the region of the genome. If both analyses identify a certain region of the genome as a possible protein-coding region, then the region is more likely to be correctly identified as such It is possible that each analysis may not have enough hits to be clearly distinguished from the background but because multiple analyses indicate the same region, it can still be identified as the protein-coding region.

In a particular application, a combined search could be implemented where a search is trypsin and the hits are tallied to each segment then a search is carried out with other enzymes and hits are tallied to each segment. Finally, the hits to each segment from the two searches are summed to give a composite score per segment. Only hits that are in the same frame are summed. This combined approach would dramatically increase the sensitivity of identification.

It is also possible to take missed cleaved peptides and modified peptides into account. When the cleavage rules are used to determine the theoretical peptides, the sequence of peptides resulting from a missed cleavage can also be calculated. This allows the mass of these peptides to also be determined. During the application of the method of the present invention these masses can also be compared to experimental masses. Similarly, one can calculate the mass of a modified form of each of the peptides and check these masses also when comparing against the experimental masses.

The method can be automated by writing an application or Script to take a series of peak lists and submit each in turn to a search against the genome. The results of this search can be databased and reviewed at a later time to determine the correct hit.

The present invention works particularly well with small genomes such as bacterial and yeast genomes or other eukayote genomes that have few introns and small amounts of non-coding DNA.

The method can also be used for the detection of pseudo genes which are versions of genes which have become defunct and identifying “protein families” of similar proteins. When a protein from a family of proteins is detected, a number of regions having a large number of matches may be identified. This indicates that the proteins may be members of the same protein family which may be for example be expressed in different tissues.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of identifying one or more proteins in an unannotated DNA sequence, the method comprising:
(a) dividing the DNA sequence into a plurality of sequence fragments each fragment being of substantially the same length and from about 300 to 5000 base pairs long;
(b) performing a six frame translation of each of the DNA sequence fragments to obtain six translated amino acid sequence fragments for each DNA sequence fragment;
(c) subjecting each of the translated sequence fragments to theoretical digestion to obtain a plurality of cleaved peptide sequences; and
(d) comparing experimental empirical data for peptide fragments from a protein digested in the same manner as the theoretical digestion at step (c) with the theoretical data generated in step (c) for each of the translated sequence fragments to identify one or more translated sequence fragments which include a significant number of peptides present in the digested protein.

2. The method of claim 1 wherein the step (a) of dividing the DNA sequence into a plurality of sequence fragments is performed before the step (b) of performing the six frame translation.

3. The method of claim 1 wherein the step (a) of dividing the DNA sequence into a plurality of sequence fragments is performed after the step (b) of performing the six frame translation.

4. The method of claim 1 wherein theoretically generated peptide masses are compared to the masses of the peptides experimentally generated by the digested protein and the sequence fragment which has the greatest number of theoretical peptide masses correlating to the empirical data is identified as indicating the likely location of the protein of interest in the DNA sequence.

5. The method of claim 1 wherein the masses of the peptides experimentally generated from the digested protein are determined by mass spectrometry.

6. The method of claim 1 wherein the DNA sequence is duplicated into a duplicate and an original and the original and duplicate are split in such a manner that the sequence fragments from the duplicate overlap divisions in the original genome sequence.

7. The method of claim 1 wherein the sequence fragments are from 800 to 1200 base pairs long.

8. The method of claim 7 wherein the sequence fragments are around 1000 to 1050 bases long.

9. The method of claim 1 wherein steps (c) and (a) are performed twice using different enzymes and data from the two digests is combined and analysed to identify the protein coding region of interest.

10. The method of claim 1 wherein the in theoretical digest of step (c) all theoretical peptides which contain a stop codon are discarded.

11. The method of claim 1 wherein the fragments are numbered so that an overlapping fragment is numbered n where the fragments it overlaps are numbered n−1 and n+1, where n is an integer.

12. A method of identifying one or more proteins in unannotated DNA sequence, the method comprising:
(a) performing a six frame translation of a DNA sequence to provide six translated amino acid sequences;
(b) dividing the six translated amino acid sequences into a plurality of fragments, each fragment comprising 100-1666 amino acids;
(c) subjecting each of the fragments to theoretical digestion to obtain a plurality of cleaved peptide sequences; and
(d) comparing experimental empirical data for peptide fragment for peptide fragments from a protein digested in the same manner as the theoretical digestion at step (c) with theoretical data generated in step (c) for each of the fragments to identify one or more fragments which include a significant number of peptides present in the empirically digested protein.

13. The method of claim 12 wherein each six translated amino acid sequences is duplicated into an original and a duplicate copy and the original and duplicate of each are split in such a manner that the sequence fragments from the original overlap divisions in the original sequence.

14. The method of claim 12 wherein theoretically generated peptide masses are compared to the masses of the peptides experimentally generated by the digested protein and the sequence fragment which has the greatest number of theoretical peptide masses correlating to the empirical data is identified as indicating the likely location of the protein of interest in the DNA sequence.

15. The method of claim 12 wherein step (c) is performed twice using different enzymes and data from the two digests is combined and analysed to identify a protein coding region of interest.
